(19) United States
(12) Patent Application Publication
(10) Pub. No.: US 2006/0293667 A1
(43) Pub. Date: Dec. 28, 2006

(54) Bone implant device and methods of using same

(76) Inventors: Agnes Vignery, Branford, CT (US); Nozer M. Mehta, Randolph, NJ (US); James P. Gilligan, Union, NJ (US)

Correspondence Address: OSTROLENK FABER GERB & SOFFEN, 1180 AVENUE OF THE AMERICAS, NEW YORK, NY, 100368403, US

(21) Appl. No.: 11/430,752

(22) Filed: May 9, 2006

Related U.S. Application Data
(60) Provisional application No. 60/682,456, filed on May 19, 2005.

(51) Int. Cl. A61F 2/30

(52) U.S. Cl. 606/62

(57) ABSTRACT

A bone implant device comprising a plurality of interconnected plate members, at least one plate member defining a plurality of apertures therein adapted for permitting bone growth there through from an interior portion of the bone in which the device is implanted for aiding in securing the device within the interior portion, and wherein the device is configured and adapted to minimize contact with an interior surface of the bone. The device is useful in applications including, but not limited to, repairing bone fractures, modeling bone in growing subjects, service as artificial joints and anchoring prostheses, such as an artificial limb, to a bone stub in the body of a subject.

### CROSS-REFERENCE TO RELATED APPLICATION

This application claims the benefit of the priority of Provisional Application No. 60/682,456 filed May 19, 2005, the contents of which are specifically incorporated by reference herein.

### BACKGROUND OF THE INVENTION

1. Field of the Invention

The invention relates to a bone implant device as well as to methods for its use. More particularly, the invention is directed to such a device having minimal contact with an inner surface of a bone marrow cavity wherein it is installed, while yet securely supporting, for example, another bone portion or fragment, or a prosthetic device or limb, to which the device is also connected.

2. Description of the Prior Art

Bone implants are frequently inserted into the skeletal structure of humans, particularly for, but not limited to, uses such as repairing bone fracture(s) and other bone trauma, for joint (e.g., hip, shoulder, knee, ankle, etc.) replacement, and for attaching prosthetic devices and/or limb(s) to the skeleton. One major difficulty associated with the installation of such bone implants, however, is to ensure their attachment to the adjacent skeletal bone. Clearly, in those situations in which permanency is necessary or desirable, the implanted device should remain permanently adhered to the contacting bone surface. There are several common prior art methods by which such devices have been attached to the bone: (1) force-fitting the device into the medullary canal of the bone; (2) securing the device in the bone with the use of screws or pins; (3) bonding the device to the bone by the use of a “bone cement”; and (4) providing the device with a fenestration (or opening) into which bone may grow, or providing on the surface of the device a porous coating layer which serves the same purpose.

Force or friction fitting a bone implant device into the bone canal provides a very stiff overall structure capable of loosening over time. Moreover, the insertion technique often tends to damage or destroy osteoblasts located around the inner surface of the medullary canal, thus preventing or at least suppressing the very bone formation needed for securing the implant within the bone.

The use of screws and/or pins as the sole means for retaining such devices in position may lead to additional complications. Failure of a single screw or pin, e.g., while the limb containing the bone is in use following the surgery, requires a further surgical procedure to repair or entirely replace the insert since the lack of any other fixation means results in a device which is unable, under such circumstances, of performing its intended function. This is, of course, highly undesirable.

The use of bone cements creates additional difficulties. While such cements do tend to provide the initial fixation necessary to permit healing following surgery, their use typically results in a very stiff overall structure, which is often prone to loosening over time. Furthermore, their presence may provoke tissue reactions in individuals sensitive to the composition of these materials.

One example of a bone insert device comprising a fenestra or opening is shown in U.S. Pat. No. 3,228,393 to Michele. The device is provided with two such openings (30,31) but it is required that the openings be packed with bone grafts (32, 33) prior to installation of the device within the bone. U.S. Pat. No. 3,228,393 describes providing a portion of an insert with fenestrae. Bone grafts pass through the fenestrae and cancellous bone is packed into the remaining recesses and grooves in the fenestrae to eventually unite with the cortical bone of the grafts. Another arrangement is shown in U.S. Pat. No. 6,758,849 to Michelson, which is directed to interbody spinal fusion implants. In one embodiment, the device forms a chamber defining a plurality of openings therein, wherein the chamber can be filled with and hold any natural or artificial osteoconductive, osteoinductive, osteogenic or other fusion enhancing material.

Each of the above-described methodologies requires lengthy and relatively difficult procedures in the midst of the implant operation, dealing with the introduction and packaging of foreign bone matter into the patient's bone structure. There is also the question of histocompatability of the added bone matter with the existing bone.

Implants having porous surface coatings rely for fixation on the ingrowth of bone or other connective tissue into the coating on the surface of the implant device, thereby anchoring the device to the bone. Examples of such arrangements are found in, for example, U.S. Pat. No. 3,605,123 to Hahn and U.S. Pat. No. 5,489,306 to Gorski. A number of technical concerns are encountered with the use of such devices, however, in choosing appropriate coating materials and in determining the proper method(s) for the application of these materials to the implant device. Not all of these concerns have yet been resolved.

There has thus been a long felt need for an easily installed, yet readily secured, bone implant device providing sufficient strength for use in, e.g., repairing fractures and other bone trauma, serving as an artificial joint and acting to secure a prosthetic limb to the skeletal structure, wherein the device is configured and adapted to achieve a reduced amount of contact with the inner surface of the bone marrow cavity, i.e., the endosteum, to facilitate bone formation by osteoblasts lining the bone marrow cavity adjacent the surface of the bone implant device. The present invention is believed to adequately meet, if not surpass, each of these requirements.

### SUMMARY OF THE INVENTION

It is an object of the present invention to provide an apparatus and a method for facilitating bone growth in instances including, but not limited to, the repair of bone fractures or other bone trauma. The apparatus or device possesses the dual properties of providing a rigid structure, while also providing minimal contact (as that term is defined below) with the medullary canal within the bone, thus permitting bone growth to occur with a minimal amount of impedance. The apparatus is capable, moreover, of a variety of additional uses including, but not limited to, methods for modeling bone growth in growing subjects and for anchoring prosthetic appendages to the body of a subject.

In one embodiment the invention is directed to a bone implant device comprising a plurality of interconnected plate members. At least one such plate member defines a plurality of apertures adapted to permit bone growth through the aperture from an interior portion of a bone in which the device is implanted. This aids in securing the device within the interior of the bone. Moreover, the device is configured and adapted to minimize contact with the interior portion of the bone surface, thus facilitating bone growth due to osteoblasts lining the bone marrow cavity. The device is thus configured and adapted to reduce, as much as possible, contact with the inner bone surface which would otherwise serve to hinder or prevent bone formation by osteoblasts lining that bone surface, while still retaining a required amount of strength and rigidity for the device to serve its intended purpose. The need for maintaining a balance between the opportunity for growing new bone which serves to “lock” the device within the bone marrow cavity, and maintaining a required degree of strength and rigidity of the device, will be well understood by one of ordinary skill in this art.

The invention is additionally directed, in another embodiment, to a method for repairing a bone fracture in a subject in need of such repair. The method comprises sufficiently stabilizing a fractured portion of a bone of the subject for a time sufficient to permit bone growth to repair the fracture, wherein the fractured portion is stabilized by inserting into a region adjacent the fractured portion a bone implant device according to the invention. The device, which is described further herein, comprises at least two interconnected plate members, wherein at least one plate member defines a plurality of apertures adapted to permit bone growth through the aperture from an interior portion of the fractured bone. This arrangement aids in securing the device within the bone. The device is configured and adapted to minimize contact with the interior surface of the bone, such that no more than 75% of the bone's inner surface is in contact with the bone's inner surface following its installation, to thus aid in preventing suppression or reduction of bone growth by osteoblasts located within the marrow cavity and/or along the inner bone surface.

In a further embodiment the invention concerns a method for modeling bone growth in growing subjects. The method comprises causing a growing portion of at least one bone to grow into a desired shape or length. This is achieved by inserting within the bone portion a bone implant device comprising a plurality of interconnected plate members. At least one plate member defines a plurality of apertures adapted to permit bone growth through the aperture from an interior portion of the bone in which the device is implanted. This aids in securing the device within the bone. The device is configured and adapted to minimize contact with the interior surface of the bone, such that no more than 75% of the bone's inner surface is in contact with the device following its installation, to aid in preventing suppression or reduction of bone growth by osteoblasts located within the marrow cavity and/or along the inner bone surface.

In a still further embodiment the invention is directed to a method for anchoring a prosthetic appendage to the body of a subject. The method comprises inserting a first end portion of a bone implant device within a bone stub remaining at a location where the prosthesis is to be anchored. The bone implant device comprises a plurality of interconnected plate members. At least one plate member defines a plurality of apertures adapted to permit bone growth through the aperture from an interior portion of the bone stub in which the device is implanted. The device is configured and adapted to minimize contact with the interior portion of the bone stub, such that no more than 75% of the bone's inner surface is in contact with the device following its installation, to assist in preventing suppression or reduction of bone growth by osteoblasts located within the marrow cavity of the stub and/or along the periphery of the inner bone surface. The prosthesis is secured to a second, opposed end portion of the bone implant device.

Another embodiment of the invention is directed to a method for securing a bone implant device within an interior portion of a bone of a subject. The method comprises: (a) locating at least a portion of a bone implant device within a bone marrow cavity of the bone, the bone implant device comprising a plurality of interconnected plate members, wherein at least one such plate member defines a plurality of apertures adapted for permitting bone growth therethrough from an interior portion of the bone marrow cavity, for aiding in securing the device within the bone, wherein the device is configured and adapted to minimize contact with the interior portion of the bone, i.e., such that no more than 75% is in contact with the device following its installation; (b) mechanically inducing an increase in osteoblast activity in the subject; and (c) elevating the blood concentration of at least one bone anabolic agent in the subject, wherein steps (b) and (c) above are performed in any order, but in sufficient time proximity that the elevated concentration and the mechanically induced increase in osteoblast activity at least partially overlaps.

A further embodiment of the invention involves an alternate method for securing a bone implant device within the interior bone portion. The method comprises: (a) locating at least a portion of a bone implant device within a bone marrow cavity of the bone, the bone implant device comprising a plurality of interconnected plate members, wherein at least one such plate member defines a plurality of apertures therein adapted for permitting bone growth therethrough from an interior portion of the marrow cavity for aiding in securing the device within the bone, wherein the device is configured and adapted to minimize contact with the interior surface of the bone, such that no more than 75% of the bone's inner surface is in contact with the device following its installation; (b) mechanically inducing an increase in osteoblast activity in the subject; and (c) administering to the subject at least one agent that causes elevated blood levels of an endogenous bone anabolic agent within the subject, wherein steps (b) and (c) above are performed in any order, but in sufficient time proximity that the elevated concentration of the anabolic agent and the mechanically induced increase in osteoblast activity at least partially overlaps.

Other features and advantages of the present invention will become apparent from the following description of the invention which refers to the accompanying drawings.

### BRIEF DESCRIPTION OF THE DRAWINGS

FIG. 1 is a sectional view of a prior art bone implant device whereby a solid rod is frictionally inserted within the bone marrow cavity;

FIG. 2 is a plan view of two slotted plate members adapted for producing, following their interconnection, a bone implant device according to one embodiment of the invention;

FIG. 3 is a sectional view through a bone having implanted therein a bone implant device according to the invention formed by interconnecting the two slotted plate members shown in FIG. 2;

FIG. 4a is a perspective view of a bone implant device formed using three plate members connected to one another via hinge members according to another embodiment of the invention;

FIG. 4b is a sectional view through a bone having implanted therein a bone implant device comprised of three plate members, illustrating one possible arrangement of the bone implant device;

FIG. 4c is a sectional view through a bone having implanted therein a bone implant device comprised of three plate members, illustrating an alternate arrangement of the bone implant device of the invention;

FIG. 5 is a sectional view through a bone having implanted therein a bone implant device comprised of four plate members according to a further embodiment of the invention;

FIG. 6a is a sectional view through a bone having implanted therein a bone implant device according to the invention comprising a centrally positioned tubular plate member having a plurality of adjustable substantially planar plate members extending therefrom;

FIG. 6b is a sectional view through a bone having implanted therein a bone implant device according to the invention comprising a centrally located tubular plate member having a plurality of fixed substantially planar plate members extending therefrom;

FIG. 6c is a sectional view through a bone having implanted therein a bone implant device according to the invention comprising a centrally located tubular member surrounded by a single helical plate member which extends outwardly from the central member;

FIG. 7a is a perspective view, partially in section, illustrating a bone implant device according to the invention used in forming an artificial joint;

FIG. 7b is a sectional view taken along the line 7b-7b of FIG. 7a;

FIG. 8a is a perspective view, partially in section, illustrating a bone implant device according to the invention used in joining a prosthetic limb to an existing bone stub; and

FIG. 8b is a sectional view taken along the line 8b-8b of FIG. 8a.

### DETAILED DESCRIPTION OF PREFERRED EMBODIMENTS OF THE INVENTION

In one embodiment the invention is directed to a bone implant device comprising two or more interconnected plate members. At least one plate member defines a plurality of apertures adapted to permit bone growth through the aperture from an interior portion of a bone in which the device is implanted. The number and arrangement of these apertures is an important consideration in fabricating the plate members of the invention and the appropriate choices involving these parameters can be readily made by those having ordinary skill in this art. Bone growth through the apertures aids in securing the device within the bone marrow cavity. The device is configured and adapted to minimize contact with the interior portion of the bone to aid in preventing suppression or reduction of bone growth by osteoblasts located within the marrow cavity and/or along the inner bone surface. As used herein, the phrase minimize (or minimal) contact is defined to mean that no more than 75% of the bone's inner surface, i.e., the endosteum, is in contact with the device following its installation within the marrow cavity of a bone. In alternate embodiments of the invention, however, the degree of contact may be any percentage less than 75%. For example, the degree of contact between the device and the bone inner surface may amount to no more than 70%, 65%, 60%, 55%, 50%, 45%, 40%, 35%, 30%, 25%, 20%, 15% or 10%. These values are provided only as examples, however and should not necessarily be construed as specific limitations upon the invention, so long as the percentage of contact is no more than 75%.

The device may optionally be at least partially additionally secured within the bone marrow cavity with the use of at least one fastening device. Useful fasteners include, but are not limited to, screws, nails or clips. Alternate fastening devices capable of performing the same securing function are also considered as falling within the scope of the invention. In a further alternate embodiment, a bicompatible adhesive may be utilized to secure, or further secure, the device within the bone cavity.

The plate members of which the device is comprised are optionally interconnected at a predetermined angle to one another. The angle is chosen to correspond with the configuration of the space available within an interior bone portion, i.e., the bone marrow cavity, of a subject wherein the device is to be inserted and affixed. The chosen angular orientation may be maintained by, for example, one or more adjustment device(s) located on at least one of the plate members. Representative adjustment devices include, but are not limited to, clips, clamps, detents and the like.

In one embodiment, one or more of the plate members define at least one slotted aperture (see, e.g., FIG. 2). The slotted aperture(s) may be configured and adapted to permit an interlocking fit between the slotted plate member and at least one additional plate member which may, if desired, be provided with a corresponding slotted aperture as well. The device, in a related embodiment, thus may be comprised of first and second interconnected plate members, wherein the plate members are interconnected by insertion of a portion of the first plate member, whether slotted or not, into a slotted aperture in the second plate member.

In an alternate arrangement, at least two plate members may be interconnected by a hinge member joining an edge portion of the plate members. Each hinge member forms an axis of rotation of the plate members connected thereby. In a related embodiment, the device may, for example, be comprised of first and second interconnected plate members, wherein the plate members are interconnected by a hinge member along an edge portion thereof, with the hinge member forming an axis of rotation of the plate members. In a still further embodiment, the device may comprise three (3), four (4), or more plate members wherein at least two of the plate members are interconnected to an adjacent member by a hinge member along an edge portion thereof, and wherein the hinge member forms an axis of rotation of the plate members which are interconnected thereby.

In one embodiment, the plates of which the device is comprised are substantially planar in shape. Alternately, at least one plate member may be at least partially curvilinear in shape. Still further, at least one said plate member may, if desired, be curved into an open tubular shape. Thus, as used herein the terms “plate” and “plate member” are not to be construed solely as referring to members having a substantially flat outer surface. That is, in accordance with the present invention one or more of the plate members used in forming the bone implant device of the invention may be at least partially curved, even to the point wherein at least one member is curved completely around so that its opposed edges come into contact with each other, thus forming an open tubular member.

In an additional embodiment of the invention, the implant device comprises at least one plate member curved into an open tubular shape, and at least one additional plate member attached at one end thereof to an outer surface of the tubular plate member, with a second, opposed end of the additional member extending outwardly from the tubular member. In one alternative embodiment, the at least one additional plate member extending from the tubular member may be secured to the tubular member at a fixed angle to the outer surface of the tubular member. In another embodiment, however, the at least one additional plate member extending from the tubular member may be movably attached to the tubular plate member and thus may extend outwardly at an adjustable angle to the outer surface of the tubular member.

In a further embodiment of the invention, at least one plate member of the implant device may extend outwardly from an inner portion of a tubular plate member through an aperture in the tubular member, wherein the aperture through which the plate member extends is configured and adapted to permit passage of the extending plate member at that location at least partially into and out of, i.e., through, the tubular plate member. This arrangement serves to permit adjustment of the distance the extending member extends outwardly from the tubular plate member to take into account, for example, bones having relatively small or irregularly shaped marrow cavities, thus permitting a customized fit of the implant device therein. The outwardly extending plate member may, if desired, be provided with an adjustment mechanism for controlling and/or adjusting the distance the extending plate member extends outwardly from the tubular plate member. The adjustment mechanism may comprise, for example, at least one detent or clip.

In a preferred embodiment a tubular plate member of the bone implant device is provided along its outer surface with at least one outwardly extending member having a helical configuration. The helical member may be formed integral with or else be detachably secured to the tubular plate member. In a particular embodiment, the tubular plate member is provided with a single, continuous outwardly extending plate member on its outer surface, wherein the outer plate member has a helical configuration. The embodiment is preferable in that it provides what is believed to be the greatest amount of rigidity to the bone in which it is inserted, corresponding with the least practical amount of contact between the device and the bone.

The plate members of the invention may be formed of a variety of materials including, but not limited to, various metals, ceramics, plastics, carbon composites and resins. Useful metals include, but are not limited to, stainless steel, titanium and alloys of cobalt and chrome. In one embodiment, the plates are formed of a strong, durable, non-rusting metal, such as titanium. A non-exclusive example of a useful plastic is polyethylene. Various combinations of materials may also be used in forming devices according to the invention including, as a non-limiting example, composites comprising one or more ceramics combined with one or more plastic polymers. One non-limiting example of such a composite is sold under the name PLASTI-BONE® by Advanced Ceramic Research, Inc. located in Tucson, Ariz. U.S.A. In choosing an appropriate material of which to form the device of the invention, it is important to keep in mind that the material must be bio-compatible, as well as resistant to corrosion, degredation and wear. In an additional embodiment of the invention, however, the device may be formed of a biodegradable material, chosen or adapted to bio-degrade after a predetermined length of time, e.g., the time needed for repair of a fracture in a fractured bone. The gradual breakdown or elimination of the device would also serve, moreover, to provide additional room within the marrow cavity for further bone growth. In further embodiments, the device could be formed, for example, of an osteo-conductive material, adapted to direct the formation of bone in, e.g., a predetermined direction, and/or an osteo-inductive material adapted to induce, e.g., additional and/or faster, bone formation. Still further, the device could be comprised of elements formed from more than one of the materials described above, i.e., a biodegradable material, an osteo-conductive material and/or an osteo-inductive material. One of ordinary skill in this field of art would be readily able to determine the proper material for use in forming the plate members of which the device is comprised.

In an additional embodiment at least one plate member is at least partially coated, or at least partially impregnated, with a bone anabolic agent for promoting bone growth within the interior portion of the bone within which the device is implanted. The bone anabolic agent may be, but is not necessarily, selected from the group consisting of a parathyroid hormone (PTH) or truncate thereof, in the free acid or amide form, anabolic Vitamin D analogs, a low-density lipoprotein receptor-related protein 5 (LRP5), an activator of non-genomic estrogen-like signaling (ANGELS), a bone morphogenic protein (BMP), an insulin-like growth factor (IGF), a fibroblast growth factor (FGF), sclerostin, leptin, a prostaglandin, a statin, strontium, a growth hormone, a growth hormone releasing factor (GHRF), hepatocyte growth factor (HGF), calcitonin gene related peptide (CGRP), parathyroid hormone related peptide (PTHrP), Transforming Growth Factor (TGF)-β1 and combinations thereof. Alternately, the growth factors described above can be used to coat the surface of the device by a technique known as Surface Induced Mineralization (see, e.g., Voelker, JAMA (1998), vol. 280, p. 315, incorporated herein by reference).

In a particular embodiment of the invention the bone anabolic agent may be at least one parathyroid hormone. The parathyroid hormone may be one selected from the group consisting of natural parathyroid hormone, truncated natural parathyroid hormone in the free acid form, an amidated truncate of natural parathyroid hormone and combinations thereof. Truncates of natural parathyroid hormone useful with the invention include, but are not limited to, PTH[1-30], PTH[1-31], PTH[1-32], PTH[1-33], and PTH[1-34], in the free acid or amide form, and combinations thereof. PTH[1-34]OH and PTH[1-34]NH₂ have been found to be particularly useful in the invention.

In an alternate embodiment at least one plate member of the bone implant device of the invention is at least partially coated, or at least partially impregnated with an agent that causes an increased expression of an endogenous bone anabolic agent into the blood of a subject within which the device is implanted. In one embodiment the at least one plate member is at least partially coated or impregnated with a calcilytic agent applied in therapeutically useful amounts. Alternately, however, if desired, the bone anabolic agent and/or an agent causing increased expression of endogenous bone anabolic agent may be directly administered, in a systemic fashion, to the patient, rather than being coated on or impregnated into, the plate members. The methodologies, dosages, etc. relating to such administration are as described below in the discussion of an embodiment of the invention incorporating the mechanical inducement of osteoblast activity.

The number and arrangement of the apertures adapted for permitting bone growth, i.e., to “lock” the implant device within the bone, may vary widely depending upon the intended application for the device. Either or both the number of apertures and/or their distribution upon one or more plate members of the device may be altered as necessary to take into account a particular use to which the device is to be put. There is, however, a well-understood trade off between aperture volume (i.e., empty space) and plate member strength. That is, increasing the amount of empty space defined by a plate member by, for example, increasing the number and/or size of the apertures, will likely result in a loss of plate strength unless a corrective measure, such as thickening the web portion of the plate or forming the plate member from a material having an enhanced weight-bearing capability and/or resistance to stresses induced by twisting, etc., is undertaken so as to permit a subject to carry out their normal everyday activities. Additionally, the device must possess sufficient strength to permit it to be wedged, without damage thereto, within a subject's bone marrow cavity during installation. As a non-limiting guideline, the device should possess the following degree of compressive strength (as measured in MegaPascals, i.e., MPa's) depending upon the bone in which it is to be installed: (1) in the femur, the device should provide a compressive strength of at least about 167 MPa; (2) in the humerus, at least about 132 MPA; (3) in the radius, at least about 114 MPA; (4) in the tibia, at least about 159 MPA; (5) in the neck, at least about 10 MPA; and (6) in the lumbar vertebrae, at least about 5 MPa.

The accompanying drawings, which are incorporated into and constitute part of, this specification, illustrate a variety of features and embodiments of the bone implant device according to the invention and, together with the description provided herein, they serve to explain the principles of the invention. It is to be understood, of course, that both the drawings and the description are explanatory only and are not restrictive of the invention.

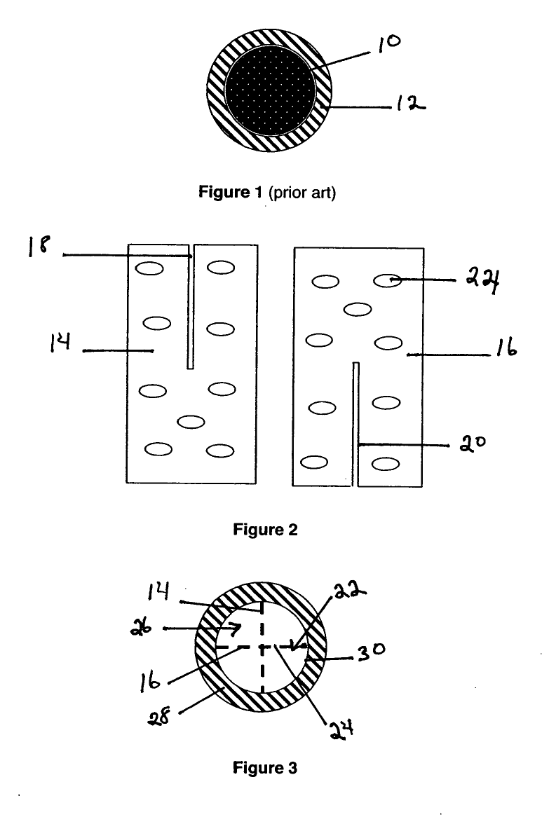

FIG. 1 provides, for comparison purposes with the present invention (as shown in the remaining figures), an illustration of a bone implant device constructed according to the prior art wherein the device comprises a substantially solid rod member 10, which is force or friction fit into the medullary cavity (not shown) of a bone 12. As noted above, prior art devices of this type suffer from a variety of inadequacies, not least of which is the tendency of the device to loosen upon use of, for example, a limb incorporating the bone following implantation of the device, and the damage such frictional insertion typically causes to the osteoblasts remaining in the medullary canal when the solid rod is jammed therein. The reduction or total destruction of such osteoblasts thus significantly decreases the amount of bone formed around the outer periphery of the implant, which bone growth is vitally important for maintaining the implant in position within the bone cavity.

In FIG. 2 is shown two substantially planar plate members 14, 16 which may be interconnected via slotted apertures 18, 20 to form bone implant device 22 as shown in FIG. 3. Plate members 14 and 16 are each provided with a plurality of apertures 24. Apertures 24 are configured and adapted to permit bone produced by osteoblasts remaining in the medullary cavity upon installation of the device to grow therethrough to the opposed side(s) of the plate member(s), thus locking device 22 within the bone interior. Moreover, the width of slotted apertures 18, 20 may be varied as desired to provide a degree of “play” in the angle between plate members 14 and 16. This is to enable the surgeon installing the device to take into account the amount and configuration of the space available within the inner, i.e., bone marrow, cavity 26 of bone 28 for installation of device 22.

FIG. 3 shows device 22 implanted within the cavity 26 of a bone 28. As may be clearly seen in, for example, FIG. 3, device 22 is configured and adapted to minimize (i.e., no more than 75%) contact between plates 14,16 comprising the device and the interior surface 30 of bone 28. This permits osteoblasts located adjacent surface 30 to be subsequently converted to bone which grows through apertures 24, thus locking device 22 in place within bone 28

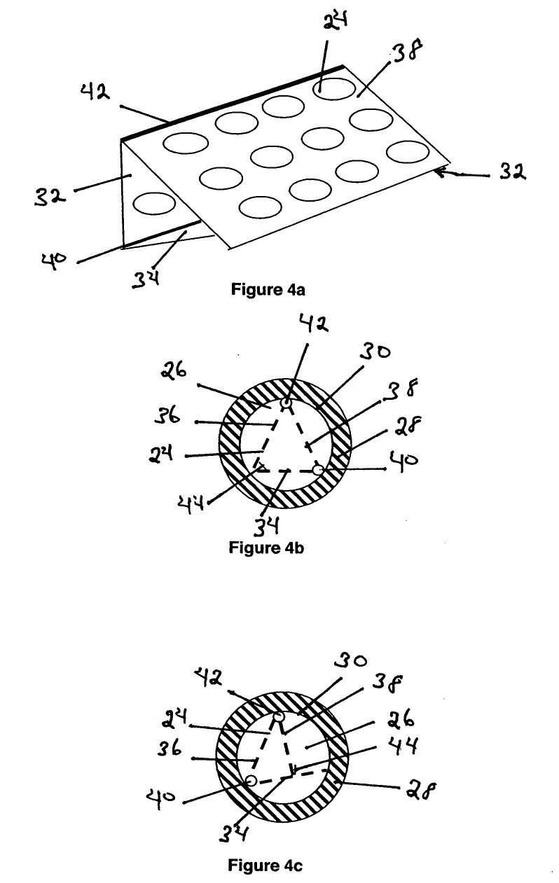

FIG. 4a shows a bone implant device 32 produced according to the invention wherein device 32 is comprised of three substantially planar plate members 34, 36,38 substantially similar in appearance to plate members 14, 16 shown in FIG. 2, except that slotted apertures 18, 20 may optionally be dispensed with as plate members 34, 36, 38 are joined together by hinge members 40,42 located along their opposed longitudinal edges. Hinge members 40, 42 form an axis of rotation for plate members 34,36,38. At least one said plate member is provided with one or more aperture(s) 24 which serve, as noted above in the discussion of FIG. 2, to permit bone growth therethrough which serves to lock device 32 within a bone in which it is installed.

FIG. 4b illustrates an embodiment wherein device 32 is installed within bone cavity 26 in a triangular orientation, providing a great deal of strength to device 32 while minimizing contact between device 32 and inner bone surface 30. The plate members may optionally be rigidly connected but, as shown, they also may be connected by hinge members 40, 42 to permit relative movement of each plate member toward and away from the other plate members. A device such as a clip or a detent 44 may be utilized to maintain plate members 34,36,38 in a desired orientation.

FIG. 4c illustrates a variation in the arrangement shown in FIG. 4b, wherein one “leg” of the triangle, formed by plate member 38, is rotated slightly inwardly toward plate member 36 via hinge member 42 and maintained in the indicated position through the use of, e.g., a clip or detent 44.

FIG. 5 illustrates a further variation of the invention wherein a bone implant device 46 is formed of four plate members 48,50,52,54. The plate members may, if desired, be rigidly connected. As shown, however, plate members 52,54 are connected via hinge member 56; plate members 54 and 48 via connected hinge member 58; and plate members 48 and 50 are connected via hinge member 60.

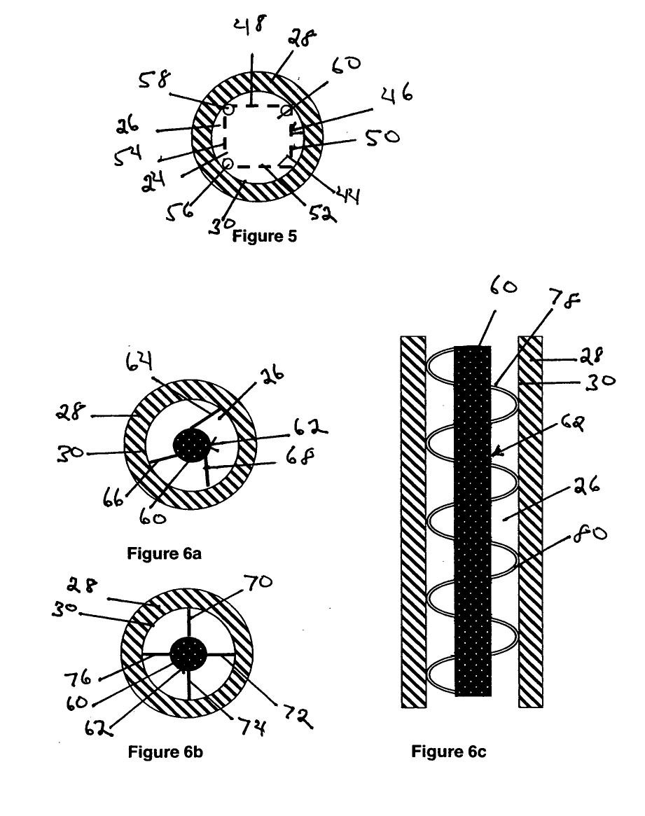

FIGS. 6a, 6b and 6c illustrate alternate embodiments of the invention comprised of a central tubular shaped plate member 60 defining a plurality of apertures 24 (not shown) therein. Extending from the outer surface of tubular member 60 is one or more plate member(s) configured and adapted for contacting the inner surface 30 of bone 28 within which the bone implant device 62 of the invention is implanted. FIG. 6a illustrates one variation provided with three outwardly extending substantially planar plate members 64,66,68, one or more of which defines a plurality of apertures 24 (not shown) configured and adapted to permit bone growth therethrough for locking device 62 within bone cavity 26. Plate members 64,66, 68 may be made adjustable in that the angle between each plate member and the outer surface of tubular plate member 60 may be modified as required depending upon the amount and configuration of the space available within bone cavity 26. Still further, if desired, slots (not shown) may be provided in tubular member 60 such that plate members 64,66, 68 extend outwardly from these slots and may thus be moved inwardly or outwardly, i.e., toward or away from tubular plate member 60, respectively, as required, in order to customize the “fit” of device 62 within cavity 26. Once the proper degree of extension is determined, these moveable plates may, in one embodiment, be locked into position.

The variation shown in FIG. 6b is similar in most respects to that shown in FIG. 6a, except that the embodiment in FIG. 6b comprises four outwardly extending plate members 70,72,74,76. These four plate members may be rigidly or adjustably connected to tubular plate member 60 as described above with regard to FIG. 6a, and at least one said plate member 70,72, 74, 76 and preferably all said members, is provided with apertures 24 (not shown) configured and adapted for the purpose described above.

In the embodiment shown in FIG. 6c, the device 78 comprises a tubular plate member 60 centrally located within bone cavity 26 and at least partially surrounded by a single helically-shaped plate member 80 which winds around the outer surface of tubular plate member 60. Optionally, helical plate member 80 is also provided with apertures 24 (not shown) for permitting bone growth therethrough.

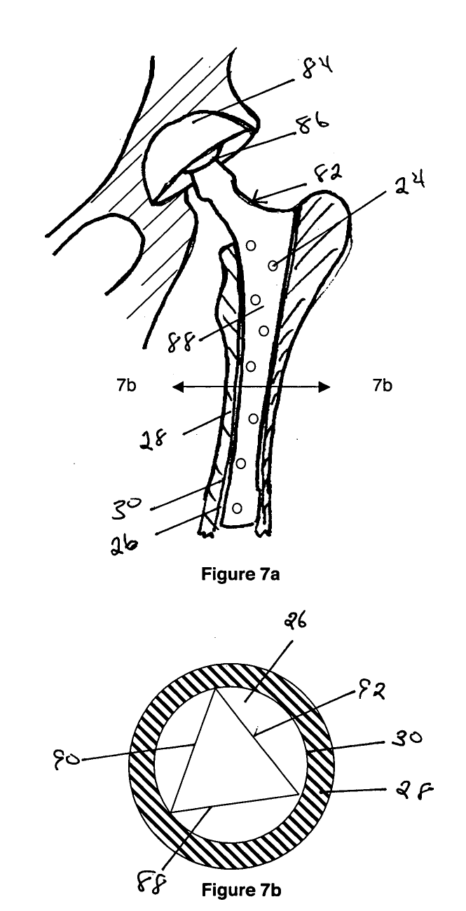

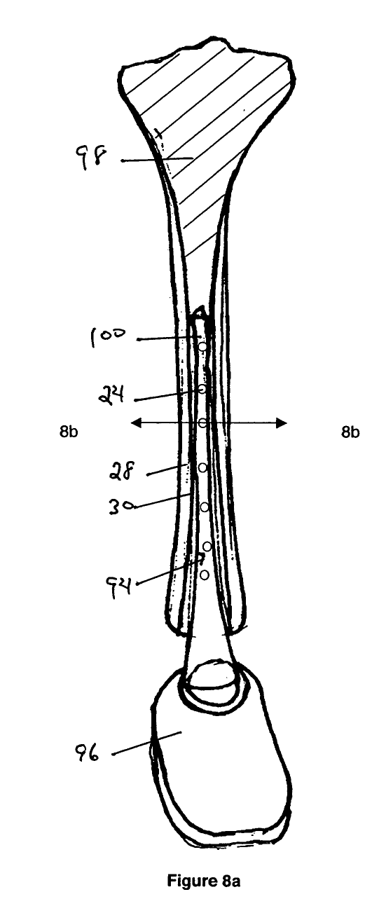

FIG. 7a,b are directed to an embodiment wherein a device 82 according to the invention is used in forming an artificial joint. The invention will work equally effectively upon use in forming hip joints, as well as joints other than hip joints, such as knee joints, shoulder joints and the like. Thus, it is only for the purpose of illustration that the artificial joint shown is a hip joint. Device 82 is comprised of acetabular head member 84 which is pivotally connected, as at 86, to a substantially triangularly-shaped structure comprised of three plate members 88,90,82 (only plate member 88 can be seen in the view shown in the Figure) which is inserted within cavity 26 formed in bone 28. At least one, and preferably all three, plate members 88,90,92 define a plurality of apertures 24 configured and adapted to permit bone growth due to osteoblasts located along the inner surface 30 of bone 28 to pass into an inner open triangular portion defined by plate members 88,90,92 of device 82.

FIG. 7b provides a cross-sectional view of device 82 taken along line 7b-7b of FIG. 7a illustrating the triangular arrangement of plate members 88,92,92. As can be seen from the figure, such a triangular arrangement minimizes contact between device 82 and the inner surface 30 of femur bone 28 in that only the “points” of the triangular shape actually contact surface 30 of the bone.

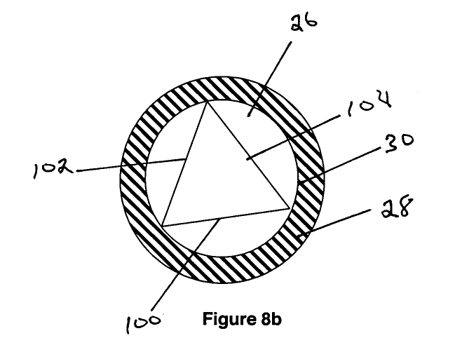

FIGS. 8a, b are directed to an embodiment wherein a bone insert device 94 according to the invention is used in joining a prosthetic limb 96 to an existing bone stub 98. Device 94 is comprised of three co-joined plate members 100,102,104 (only plate member 100 can be seen in FIG. 8a), one or more of which defines a plurality of apertures 24 as described above.

FIG. 8b provides a cross-sectional view of device 94 taken along line 8b-8b of FIG. 8a illustrating the triangular arrangement of plate members 100,102,104. As in the case of the embodiment illustrated in FIGS. 7a,b, this arrangement minimizes (no more than 75%) contact between device 94 and the inner surface 30 of bone 28.

In accordance with the description of the invention contained herein, it is important to note that the device 82 of FIGS. 7a,b and the device 94 of FIGS. 8a,b are not limited to embodiments comprised of a triangular-shaped body formed of three plate members defining a plurality of apertures configured and adapted for permitting bone growth therethrough. That is, the devices 82, 94 illustrated therein may optionally be formed using alternate embodiments of the invention, such as those illustrated in FIGS. 5 and 6a, b, c for example.

The invention is additionally directed, in another embodiment, to a method for repairing a bone fracture in a subject in need of such repair. The method comprises sufficiently stabilizing a fractured portion of a bone of the subject for a time sufficient to permit bone growth to repair the fracture, wherein the fractured portion is stabilized by inserting into a region adjacent the fractured portion a bone implant device as described herein.

In a further embodiment the invention is directed to a method for modeling bone growth in growing subjects. The method comprises causing a growing portion of at least one bone to grow into a desired shape or length. This is achieved by inserting within the bone portion a bone implant device as described herein.

In an additional embodiment the invention is directed to a method for anchoring a prosthetic appendage to the body of a subject. The method comprises inserting a first end portion of a bone implant device as described herein within a bone stub remaining at a location where the prosthesis is to be anchored, and wherein the prosthesis is secured to a second, opposed end portion of the bone implant device.

The bone implant device used in practicing the methods of the invention, including the repair of bone fractures, modeling bone growth in growing subjects and anchoring a prosthetic appendage to a subject's body, is that described above. The usefulness of the device is not limited solely to the indicated methods, however, since the skilled artisan may determine a variety of additional uses for the device according to the invention. All such uses as are encompassed within the teachings contained herein are believed to fall within the scope of the present invention.

In a further alternate embodiment, installation of a bone implant device according to the invention within a subject is coupled with a treatment of the subject which induces greater and more rapid bone formation than would otherwise normally occur, which bone formation serves, as indicated above, to secure, i.e., anchor, the bone implant device within the bone of the subject. A variety of methods are well-known in the art for fostering such bone formation. However, such methods are typically systemic in nature. That is, they treat the whole skeleton as a single entity. Certain of the methods described herein for use in the present invention, however, permit the targeting of one or more specific bones, e.g., those of the hip, shoulder, spine or wrist, which may require a more focused treatment.

In one embodiment, the treatment induces bone formation in a subject by a method which comprises the steps of (a) mechanically inducing an increase in osteoblast activity in the subject; and (b) elevating blood concentration of at least one bone anabolic agent therein, e.g., by administering such an agent or by administering a compound which causes natural formation of such an agent. Typically, the inducement step occurs prior to, or concurrent with, installation of the bone implant device. Inducement of bone growth, as that phrase is used herein, may include, for example, generating new or additional bone at locations where such bone growth is not presently taking place and/or stimulating the growth (i.e., increasing the rapidity thereof) of bone which is already in the process of formation. Administration of the bone anabolic agent may commence, if desired, prior to such installation and typically continues for a pre-determined period beyond the installation step. Steps (a) and (b) above may be performed in any order, but are to be carried out in sufficient time proximity that the elevated concentration of the anabolic agent and the mechanically induced increase in osteoblast activity at least partially overlaps. In one embodiment of the invention, therefore, the bone anabolic agent is administered to the subject contemporaneous with the mechanical inducement of osteoblast activity. In another embodiment, the bone anabolic agent is administered subsequent to such mechanical inducement. In still another embodiment, the bone anabolic agent may be administered prior to the mechanical inducement such that elevated levels of bone anabolic agent are already present at the time of mechanical inducement, which levels may then be maintained or continued intermittently for an extended period thereafter. Without being bound in any way by theory, applicants believe that the inducement of bone growth takes place due to the combined effects of (1) the mechanical inducement of osteoblast activity in the subject, coupled with (2) an elevation in the blood concentration of the at least one bone anabolic agent.

The bone in which the bone implant device of the invention is installed defines a bone marrow cavity therein. The bone marrow cavity contains, inter alia, a quantity of bone marrow and a plurality of osteoblasts. The method thus comprises mechanically altering the contents of the bone marrow cavity to thereby stimulate and thus increase osteoblast differentiation and/or activity therein. Thereafter, bone mass is increased within the cavity due to the increased osteoblast differentiation/activity. The method additionally comprises administering to the subject at least one bone anabolic agent for a duration and at a concentration sufficient to raise blood levels of the anabolic agent within the subject above natural levels thereof, and thereby prolonging the mechanically induced osteoblast activity. The mechanical alteration of the bone marrow cavity thus permits specific bone(s) of the subject, i.e., those within which the bone implant device is implanted, as well as bone(s) and/or fragment(s) located adjacent thereto, to be specifically targeted for inducing bone formation therein.

Mechanical inducement of an increase in osteoblast activity may be obtained, in one embodiment of the invention, by a process of bone marrow irrigation and ablation. Again, without being bound in any way by theory, applicants believe that the bone marrow irrigation and ablation process leads to the formation of a clot within the bone marrow cavity which, through a cascade of biochemical reactions, contributes to increasing osteoblast activity in the subject.

In another embodiment, the increased osteoblast activity may alternately be obtained by coupling the mechanical inducement with an additional form of inducement, such as biochemical inducement. Such biochemical inducement may be obtained by administering to the subject, for example, a quantity of a blood factor such as Factor (“F”) VII, Factor VIIa or a combination thereof. Following tissue or vascular injury, clotting is initiated by the binding of plasma FVII/FVIIa to tissue factor (tissue thromboplastin). This complex (FVII/FVIIa+Thromboplastin) initiates a sequence of events which leads to activation of the coagulation cascade, ultimately leading to fibrin deposition and platelet activation. This complex sequence of events may contribute in part to the stimulation of osteoblasts in the bone marrow. Factors VII and VIIa are commercially available from Novo Nordisk.

The increase in osteoblast activity obtained with the use of the method of the invention may be due to a variety of factors including, but not necessarily limited to (1) osteoblast differentiation, i.e., the production of additional osteoblasts, (2) increasing the activity and/or effectiveness of osteoblasts which are already present in inducing bone formation in the subject, and (3) a combination thereof. In a preferred embodiment of the invention, the increase in osteoblast activity would include all of the above-noted functions.

A bone anabolic agent endogenously produced in the human body is PTH[1-84] in the free acid form which is naturally found in levels of less than about 8 picomoles (pmoles) per liter in the blood of a human subject. In the embodiment described above involving administration of a material causing an increased expression (i.e., above the natural level described above) of an endogenous bone anabolic agent, the material administered may, for example, be a calcilytic agent. Calcilytic agents useful with the method of the invention include, but are not limited to any agent that limits the binding of calcium to its receptor and thereby triggers the release of endogenous PTH. Examples of such calcilytic compounds are set forth in U.S. Pat. Nos. 6,362,231; 6,395,919; 6,432,656 and 6,521,667, the contents of which are incorporated herein by reference.

Both bone anabolic agents and agents causing an increased expression of bone anabolic agents, may be administered, for example, orally, intravenously, intramuscularly, subcutaneously, via implant, transmucosally, transdermally, rectally, nasally, by depot injection, or by inhalation and pulmonary absorption. In another embodiment, either medicament may be administered once as a time release formulation, a plurality of times or over one or more extended periods. It is preferred that elevated blood levels of the anabolic agent be maintained, at least intermittently, for between 14-365 days, and more preferably for between about 30-180 days, post-mechanical induction. Intermittent administration of a parathyroid hormone, e.g., PTH[1-34]NH₂, could occur once daily or once weekly, resulting in peaks of blood concentration that return to baseline levels between doses, but nevertheless result in periodic elevated blood levels of a bone anabolic agent in a manner that overlaps the elevated osteoblast activity that is initially induced mechanically, although thereafter sustained, at least in part, by the anabolic agent.

As in the description provided above, anabolic agents useful with the invention include, but are not limited to, a parathyroid hormone (PTH) or truncate thereof, in free acid or amide form, anabolic Vitamin D analogs, a low-density lipoprotein receptor-related protein 5 (LRP5), an activator of non-genomic estrogen-like signaling (ANGELS), a bone morphogenic protein (BMP), an insulin-like growth factor (IGF), a fibroblast growth factor (FGF), sclerostin, leptin, a prostaglandin, a statin, strontium, a growth hormone, a growth hormone releasing factor (GHRF), hepatocyte growth factor (HGF), calcitonin gene related peptide (CGRP), parathyroid hormone related peptide (PTHrP) and combinations thereof. As used herein the term “parathyroid hormone” includes, but is not limited to natural parathyroid hormone, a truncate of natural parathyroid hormone, an amidated truncate of natural parathyroid hormone, an amidated natural parathyroid hormone and combinations thereof.

In one embodiment of the invention the bone anabolic agent is truncated PTH[1-34] in the free acid form. This material is commercially available in an FDA-approved pharmaceutical formulation from Eli Lilly & Co. under the trade name Forteo® (teriparatide). Other desirable bone anabolic agents for use with the invention include, but are not limited to, the amidated truncates of the natural parathyroid hormones noted above, i.e., PTH[1-30], PTH[1-31], PTH[1-33], in the free acid or the amide form, PTH[1-34]NH₂ and combinations thereof. In one preferred embodiment, the bone anabolic agent is PTH[1-34]NH₂. Methods for the preparation of truncated parathyroid hormones are described in U.S. Pat. No. 6,103,495 to Mehta et al. Moreover, methodologies for amidating such truncated parathyroid hormones are provided in, for example, U.S. Pat. No. 5,789,234 to Bertelsen et al. and U.S. Pat. No. 6,319,685 to Gilligan et al. The contents of each of these patents is specifically incorporated herein by reference.

In one embodiment, a sufficient amount of amidated truncated parathyroid hormone as discussed herein is administered to a subject to achieve, and thereafter maintain, a pulsatile blood concentration thereof in the subject of between abut 50 and 350 μg/ml, preferably between about 100 and 200 μg/ml, and most preferably about 150 μg/ml. In another embodiment, the blood concentration of the parathyroid hormone in the subject is raised to its preferred level in no later than 7 days following mechanical alteration of the contents of the bone marrow cavity. As would be well known in this art, an appropriate dosage of the PTH bone anabolic agent must be calculated to achieve the above-indicated blood concentrations. In the case of injectable formulations, for example, the dose (in pure weight of active hormone) given to, for example, a human subject, may be that taught in the literature relating to the bone anabolic activity of these various agents. Such dose may, but does not necessarily, range between about 100-200 μg, given once per day, more preferably between about 20-100μg per dose and most preferably between about 20-50 μg per dose. For alternate delivery routes, i.e., other than injections, the dosage may range between about 10 μg and 10 mg. Dosage levels of injectable formulations comprising bone anabolic agents other than the above-described parathyroid hormone-based agents would be consistent with those noted above for the PTH agents.

In a still further embodiment the method of the invention additionally comprises providing the subject with an elevated blood concentration of at least one antiresorptive agent, wherein the elevated concentration is sufficient to diminish resorption of new bone growth produced due to the mechanically induced enhanced osteoblast activity described above. In one embodiment, the antiresorptive agent may be administered contemporaneous with the administration of the bone anabolic agent. In another embodiment the antiresorptive agent is administered subsequent to the administration of the bone anabolic agent. In a further embodiment, the administration of the antiresorptive agent may be commenced during administration of the bone anabolic agent and such administration may then be continued beyond the termination of administration of the bone anabolic agent. Administration of the antiresorptive agent preferably continues for at least three months and more preferably between 12-24 months.

In another embodiment of the invention, a single agent may be administered having both bone anabolic and antiresorptive properties. Examples of such materials include, but are not limited to, estrogen, strontium ranalate and selective estrogen receptor modulators (SERMS).

In an embodiment of the invention, the antiresorptive agent may be a calcitonin selected from the group consisting of human calcitonin, salmon calcitonin (“sCT”), eel calcitonin, porcine calcitonin, chicken calcitonin, calcitonin gene related peptide (CGRP) and combinations thereof. In a preferred embodiment, the antiresorptive agent is salmon calcitonin. Blood levels of calcitonin, when used as the antiresoprtion agent, preferably range between about 5-500 pg/ml, more preferably between about 10-250 pg/ml and most preferably between about 20-50 pg/ml. Moreover, human dosage levels of the subject calcitonin agents necessary to achieve the above blood levels, in the case of, e.g., injectable formulations, may be those taught in the literature relating to the use of these materials as anabolic agents. Such dose may, but does not necessarily, range between about 5-200 μg given once per day, more preferably between about 5-50 μg and most preferably 8-20 μg by weight of the pure drug, administered daily. When using alternate delivery routes, the range may vary between about 5 μg and 5 mg. Salmon calcitonin (sCT) administered by alternate routes, i.e., by nasal or oral administration, would require higher dosages than those discussed above.

Alternately, a variety of antiresorptive agents other than the calcitonins are useful in the present invention. These include, generally, hormone replacement therapy (HRT) agents, such as selective estrogen receptor modulators (SERMS), bisphosphonates, cathepsin-K inhibitors, strontium ranalate and various combinations thereof. Specific examples of additional antiresorptive agents include, but are not limited to, Premarin® available from Wyeth Laboratories, which includes estrogen as the active ingredient—a typical accepted dosage is one 0.625 mg tablet daily; (2) Actonel® available from Proctor & Gamble, which includes, as the active ingredient, risedronate sodium. A typical accepted dosage is one 5 mg tablet daily or one 35 mg tablet weekly; (3) Evista® sold by Eli Lilly & Co., which includes raloxifene HCl as the active ingredient. A typical accepted dosage of this formulation is one 60 mg tablet taken daily; and (4) Fosamax® available from Merck Pharmaceuticals, which includes alendronate as the active ingredient. Typical dosages of this material are 10 mg/day or 70 mg/week.

Except where otherwise noted or where apparent from the context, dosages herein refer to the weight of the active compounds unaffected by pharmaceutical excipients, diluents, carriers or other ingredients, although such other ingredients are typically included in the variety of dosage forms useful in the invention. Any dosage form (i.e., capsule, tablet, injection or the like) commonly used in the pharmaceutical industry is appropriate for use herein and the terms “excipient”, “diluent” and “carrier” include such non-active ingredients as are typically included, together with active ingredients, in the industry. For example, typical capsules, pills, enteric coatings, solid or liquid diluents or excipients, flavorants, preservatives and the like are included. Moreover, it is additionally noted that with respect to all of the dosages recommended herein, the attending clinician should monitor individual patient response, and adjust the dosages accordingly.

The antiresorptive agent may be administered orally, intravenously, intramuscularly, subcutaneously, via implant, transmucosally, rectally, nasally, by depot injection, by inhalation and pulmonary absorption or transdermally. Moreover, the antiresorptive agent may be administered once, a plurality of times, or over one or more extended periods.

In a still further embodiment, the invention provides a kit containing the above-described elements, i.e., one or more bone implant device(s), at least one container having therein at least one bone anabolic agent and a mechanical alteration device for altering contents of a bone marrow cavity in at least one bone in which the device is to be installed. In another embodiment, the kit may additionally comprise an evacuation device for evacuating at least a portion of the contents from the bone marrow cavity. In a further embodiment, the kit may further comprise at least one container having therein at least one antiresorptive agent.

In one embodiment of the kit, the bone anabolic agent is selected from among natural parathyroid hormone, a truncate of natural parathyroid hormone, an amidated natural parathyroid hormone, and combinations thereof. In a preferred embodiment, the bone anabolic agent is a truncate of natural parathyroid hormone. A preferred truncate for use as the agent is PTH[1-34] in the free acid form. Other preferred truncates include amidated truncates. The bone anabolic agent may, in such a case, be thus selected from among PTH[1-30]NH₂, PTH[1-31]NH₂, PTH[1-32]NH₂, PTH[1-33]NH₂, PTH[1-34]NH₂ and combinations thereof. In a specific embodiment, the bone anabolic agent is PTH[1-34]NH₂.

In an additional embodiment of the kit, the antiresorptive agent is a calcitonin selected from the group consisting of human calcitonin, salmon calcitonin, eel calcitonin, elkatonin, porcine calcitonin, chicken calcitonin, calcitonin related gene peptide (CRGP) and combinations thereof. In a particular embodiment the antiresorptive agent is salmon calcitonin.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A bone implant device comprising a plurality of interconnected plate members configured and adapted to permit at least partial insertion of the device within an interior bone portion of a subject, said interior bone portion comprising a bone marrow cavity defined by an interior bone surface, at least one said plate member defining a plurality of apertures therein adapted for permitting bone growth therethrough from within said interior bone portion, for aiding in securing the device within the interior bone portion, wherein, following installation of the device, no more than about 75% of the interior bone surface is in contact with the device.

2. The implant device according to claim 1, wherein the device is at least partially secured within said interior bone portion with at least one fastener or with a biocompatible adhesive.

3. The implant device according to claim 1, wherein said plate members are maintained at a predetermined angle to one another, said angle chosen to correspond with an amount and a configuration of a space available within said interior bone portion for implanting the device.

4. The implant device according to claim 3, wherein said predetermined angle is maintained by at least one adjustment device located on at least one said plate member, said adjustment device being selected from the group consisting of clips, clamps and detents.

5. The implant device according to claim 1, wherein at least one said plate member defines a slotted aperture therein, said slotted aperture being configured and adapted to permit an interlocking fit between said slotted plate member and at least one additional plate member.

6. The implant device according to claim 1, wherein at least two said plate members are connected by a hinge member along an edge portion of the plate members thus connected, each said hinge member forming an axis of rotation of the plate members connected thereby.

7. The implant device according to claim 1, comprising three or more said plate members, wherein at least two said plate members are connected to an adjacent member by a hinge member along an edge portion of the connected plate members, each said hinge member forming an axis of rotation of the plate members interconnected thereby.

8. The implant device according to claim 1, wherein said plate members have a shape which is selected from the group consisting of substantially planar, at least partially curvilinear, and open tubular.

9. The implant device according to claim 8, wherein said plate member has an open tubular shape and is provided with at least one additional plate member attached at one end thereof to an outer surface of said tubular plate member and having a second, opposed end extending outwardly from said tubular plate member.

10. The implant device according to claim 9, wherein at least one said additional plate member extending from said tubular plate member extends at a fixed angle or an adjustable angle to an outer surface of said tubular plate member.

11. The implant device according to claim 8, wherein the plate member has an open tubular shape and further comprising at least one additional plate member extending outwardly from an inner portion of said tubular plate member through a slot in said tubular plate member, said slot configured and adapted to permit both inward and outward passage of the outwardly extending plate member at such location at least partially into and out of an inner portion of said tubular plate member, so as to permit adjustment of a distance said additional member extends outwardly from said tubular plate member.

12. The implant device according to claim 8, wherein said plate member has an open tubular shape, and wherein said tubular plate member is provided along its outer surface with at least one outwardly extending plate member having a helical configuration.

13. The implant device according to claim 1, wherein said plate members are formed of a material selected from the group consisting of metals, ceramics, plastics, composites and resins.

14. The implant device according to claim 13, wherein at least one said plate member is formed of a metal and wherein the metal is titanium.

15. The implant device according to claim 1, wherein at least one said plate member is at least partially coated or at least partially impregnated with a bone anabolic agent for promoting bone growth within the interior portion of a bone wherein the device is implanted.

16. The implant device according to claim 15 wherein the bone anabolic agent is selected from the group consisting of a parathyroid hormone (PTH) or truncate thereof, in free acid or amide form, anabolic Vitamin D analogs, a low-density lipoprotein receptor-related protein 5 (LRP5), an activator of non-genomic estrogen-like signaling (ANGELS), a bone morphogenic protein (BMP), an insulin-like growth factor (IGF), a fibroblast growth factor (FGF), sclerostin, leptin, a prostaglandin, a statin, strontium, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof.

17. The implant device according to claim 16, wherein the bone anabolic agent is selected from the group consisting of natural parathyroid hormone, a truncate of natural parathyroid hormone, an amidated truncate of natural parathyroid hormone, an amidated natural parathyroid hormone and combinations thereof.

18. The implant device according to claim 31, wherein the bone anabolic agent is selected from the group consisting of PTH[1-30], PTH[1-31], PTH[1-32], PTH[1-33], and PTH[1-34], in the free acid or amide form, and combinations thereof.

19. The implant device according to claim 1, wherein at least one said plate member is at least partially coated or at least partially impregnated with an agent that causes increased expression of an endogenous bone anabolic agent into the blood of a subject within which said device is implanted.

20. The implant device according to claim 19, wherein at least one said plate member is at least partially coated or at least partially impregnated with a calcilytic agent.

21. The implant device according to claim 1, wherein a major portion of at least one said plate member is provided with said apertures, and wherein the device, on account of the material of which said plate members are formed and due to its interlocking construction is imparted with sufficient strength to withstand installation and normal usage of a bone in which said device is inserted without damage to such device, such that said device imparts a compressive strength of at least about 5 MPas to said bone.

22. A method for repairing a bone fracture in a subject, the method comprising: sufficiently stabilizing a fractured portion of a bone of said subject for a time sufficient to permit bone growth to repair said fracture by inserting in an interior bone portion adjacent said fracture a bone implant device comprising at least two interconnected plate members configured and adapted to permit at least partial insertion of the device within said interior bone portion, said interior bone portion comprising a bone marrow cavity defined by an interior bone surface, at least one said plate member defining a plurality of apertures therein adapted for permitting bone growth therethrough from said interior bone portion, for aiding in securing the device within the interior bone portion, wherein, following installation of the device, no more than about 75% of the interior bone surface is in contact with the device.

23. The method according to claim 22, which further comprises at least partially securing the bone implant device within the interior bone portion with at least one fastener or with a biocompatible adhesive.

24. The method according to claim 22, wherein said plate members are maintained at a predetermined angle to one another, said angle being determined to correspond to an amount and a configuration of a space available within said interior bone portion for implanting the device.

25. The method according to claim 22, which further comprises providing at least one said plate member with a slotted aperture, said slotted aperture being configured and adapted to permit an interlocking fit between said slotted plate member and at least one additional plate member.

26. The method according to claim 22, which further comprises connecting at least two of said plate members with a hinge member along an edge portion of said connected plate members, said hinge member forming an axis of rotation of said plate members connected thereby.

27. The method according to claim 22, wherein the bone implant device comprises three or more said plate members, and wherein at least two said plate members are connected to an adjacent plate member by a hinge member along an edge portion of the interconnected plate members, said hinge member forming an axis of rotation of the plate members connected thereby.

28. The method according to claim 22, wherein said plate members are formed of a material selected from the group consisting of metals, ceramics, plastics, composites and resins.

29. The method according to claim 22, which further comprises at least partially coating or impregnating at least one said plate member with a bone anabolic agent for promoting bone growth in a fractured portion of the bone.

30. The method according to claim 29, wherein the bone anabolic agent is selected from the group consisting of a parathyroid hormone (PTH) or truncate thereof, in free acid or amidated form, anabolic Vitamin D analogs, a low-density lipoprotein receptor-related protein 5 (LRP5), an activator of non-genomic estrogen-like signaling (ANGELS), a bone morphogenic protein (BMP), an insulin-like growth factor (IGF), a fibroblast growth factor (FGF), sclerostin, leptin, a prostaglandin, a statin, strontium, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof.

31. The method according to claim 22, which further comprises at least partially coating or impregnating at least one said plate member with an agent that causes increased expression of an endogenous bone anabolic agent into the blood of the subject having a bone fracture.

32. The method according to claim 31, wherein at least one said plate member is at least partially coated or at least partially impregnated with a calcilytic agent.

33. The method according to claim 22, which further comprises systemically administering to said subject, in conjunction with said stabilizing step, a pharmaceutical composition selected from the group consisting of a bone anabolic agent and an agent that causes increased expression of an endogenous bone anabolic agent into the blood of the subject.

34. A method for modeling bone in growing subjects, the method comprising causing a growing portion of at least one bone of said subject to grow into a desired shape or length by inserting within an interior portion of said growing bone portion a bone implant device comprising a plurality of interconnected plate members configured and adapted to permit at least partial insertion of the device within said interior bone portion, said interior bone portion comprising a bone marrow cavity defined by an interior bone surface, at least one said plate member defining a plurality of apertures therein adapted for permitting bone growth therethrough from said interior bone portion for aiding in securing the device within the interior portion, wherein, following installation of the device, no more than about 75% of the interior bone surface is in contact with the device.

35. The method according to claim 34, which further comprises at least partially securing the bone implant device within said interior bone portion with at least one fastener or with a biocompatible adhesive.

36. The method according to claim 34, wherein said plate members are maintained at a predetermined angle to one another, said angle being determined to correspond to an amount and a configuration of a space available within said interior bone portion for implanting the device.

37. The method according to claim 34, which further comprises providing at least one said plate member with a slotted aperture, said slotted aperture being configured and adapted to permit an interlocking fit between said slotted plate member and at least one additional plate member.

38. The method according to claim 34, which further comprises connecting at least two of said plate members with a hinge member along an edge portion of the connected plate members, said hinge member forming an axis of rotation of the plate members connected thereby.

39. The method according to claim 34, wherein the bone implant device comprises three or more of said plate members, and wherein at least two said plate members are connected to an adjacent member by hinge members along an edge portion of said interconnected plate members, said hinge member forming an axis of rotation of the plate members which are interconnected thereby.

40. The method according to claim 34, wherein said plate members are formed of a material selected from the group consisting of metals, ceramics, plastics, composites and resins.

41. The method according to claim 34, which further comprises at least partially coating or impregnating at least one said plate member with a bone anabolic agent for promoting bone growth in the bone.

42. The method according to claim 41, wherein the bone anabolic agent is selected from the group consisting of a parathyroid hormone (PTH) or truncate thereof, in free acid or amidated form, anabolic Vitamin D analogs, a low-density lipoprotein receptor-related protein 5 (LRP5), an activator of non-genomic estrogen-like signaling (ANGELS), a bone morphogenic protein (BMP), an insulin-like growth factor (IGF), a fibroblast growth factor (FGF), sclerostin, leptin, a prostaglandin, a statin, strontium, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof.

43. The method according to claim 34, which further comprises at least partially coating or impregnating at least one said plate member with an agent that causes increased expression of an endogenous bone anabolic agent into the blood of the subject.

44. The method according to claim 43, wherein at least one said plate member is at least partially coated or at least partially impregnated with a calcilytic agent.

45. The method according to claim 34, which further comprises systemically administering to said subject, in conjunction with insertion of said bone implant device, a pharmaceutical composition selected from the group consisting of a bone anabolic agent and an agent that causes increased expression of an endogenous bone anabolic agent into the blood of the subject.

46. A method for anchoring a prosthetic appendage to the body of a subject, the method comprising inserting a first end portion of a bone implant device within an interior bone portion of a bone stub remaining at a location where said prosthesis is to be anchored, said bone implant device comprising a plurality of interconnected plate members configured and adapted to permit at least partial insertion of the device within said interior bone portion, said interior bone portion comprising a bone marrow cavity defined by an interior bone surface, at least one said plate member defining a plurality of apertures therein adapted for permitting bone growth therethrough from said interior bone portion for aiding in securing the device within the interior bone portion, wherein, following installation of the device, no more than about 75% of the interior bone surface is in contact with the device, and wherein said prosthesis is secured to a second, opposed end portion of said bone implant device.

47. The method according to claim 46, which further comprises at least partially securing the first end portion of the bone implant device within said interior bone portion with at least one fastener or with a biocompatible adhesive.

48. The method according to claim 46, wherein said plate members are maintained at a predetermined angle to one another, said angle being determined to correspond to a configuration of an available space within said interior bone portion for implanting the device.

49. The method according to claim 46, which further comprises providing at least one said plate member with a slotted aperture, said slotted aperture being configured and adapted to permit an interlocking fit between said slotted plate member and at least one additional plate member.

50. The method according to claim 46, which further comprises connecting at least two of said plate members with a hinge member along an edge portion of the connected plate members, said hinge member forming an axis of rotation of said plate members connected thereby.

51. The method according to claim 46, wherein the bone implant device comprises three or more said plate members, and wherein at least two said members are connected to an adjacent member by a hinge member along an edge portion of the plate members so connected, said hinge member forming an axis of rotation of the plate members which are interconnected thereby.

52. The method according to claim 46, wherein said plate members are formed of a material selected from the group consisting of metals, ceramics, plastics, composites and resins.

53. The method according to claim 46, which further comprises at least partially coating or impregnating at least one said plate member with a bone anabolic agent for promoting bone growth in the bone.

54. The method according to claim 53, wherein the bone anabolic agent is selected from the group consisting of a parathyroid hormone (PTH) or truncate thereof, in free acid or amide form, anabolic Vitamin D analogs, a low-density lipoprotein receptor-related protein 5 (LRP5), an activator of non-genomic estrogen-like signaling (ANGELS), a bone morphogenic protein (BMP), an insulin-like growth factor (IGF), a fibroblast growth factor (FGF), sclerostin, leptin, a prostaglandin, a statin, strontium, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof.

55. The method according to claim 46, which further comprises at least partially coating or impregnating at least one said plate member with an agent that causes increased expression of an endogenous bone anabolic agent into the blood of the subject.

56. The method according to claim 55, wherein at least one said plate member is at least partially coated or at least partially impregnated with a calcilytic agent.

57. The method according to claim 46, which further comprises systemically administering to said subject, in conjunction with insertion of said bone implant device, a pharmaceutical composition selected from the group consisting of a bone anabolic agent and an agent that causes increased expression of an endogenous bone anabolic agent into the blood of the subject.

58. A method for securing a bone implant device within an interior portion of a bone of a subject, which method comprises:
(a) locating at least a portion of the bone implant device of claim 1 within a bone marrow cavity of said bone;
(b) mechanically inducing an increase in osteoblast activity in said subject; and
(c) elevating blood concentration of at least one bone anabolic agent in said subject, wherein steps (b) and (c) are performed in any order, but in sufficient time proximity that the elevated concentration of the anabolic agent and the mechanically induced increase in osteoblast activity at least partially overlaps.

59. The method of claim 58, wherein the blood concentration of the bone anabolic agent is elevated by direct administration of a bone anabolic agent to the subject.

60. The method of claim 58, which further comprises providing said subject with an elevated blood concentration of at least one antiresorptive agent, wherein said elevated concentration is sufficient to substantially prevent resorption of new bone growth produced due to said increased osteoblast activity.

61. The method of claim 60, wherein the bone anti-resorptive anabolic agent is estrogen, strontium ranalate, calcitonin or a selective estrogen receptor modulator (SERM).

62. A method of securing a bone implant device within an interior portion of a bone of a subject, which method comprises:
(a) locating at least a portion of the bone implant device of claim 1 within a bone marrow cavity of said bone;
(b) mechanically inducing an increase in osteoblast activity in said subject; and
(c) administering to said subject at least one agent that causes elevated blood levels of an endogenous bone anabolic agent within said subject, wherein steps (b) and (c) are performed in any order, but in sufficient time proximity that said elevated concentration of said anabolic agent and said mechanically induced increase in osteoblast activity at least partially overlaps.

63. The method of claim 62, wherein the agent causing an increased expression of said endogenous bone anabolic agent within said subject is a calcilytic agent.

64. The method of claim 62, wherein the bone anabolic agent is administered orally, intravenously, intramuscularly, subcutaneously, via implant, transmucosally, rectally, nasally, by depot injection, by inhalation and pulmonary absorption or transdermally.

65. The method of claim 64, wherein said administration occurs once, a plurality of times, or over one or more extended periods.

66. The method of claim 62, wherein said at least one bone anabolic agent is selected from the group consisting of a parathyroid hormone (PTH) or truncate thereof, in free acid or amide form, anabolic Vitamin D analogs, a low-density lipoprotein receptor-related protein 5 (LRP5), an activator of non-genomic estrogen-like signaling (ANGELS), a bone morphogenic protein (BMP), an insulin-like growth factor (IGF), a fibroblast growth factor (FGF), sclerostin, leptin, a prostaglandin, a statin, strontium, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof.
